(19) United States
(12) Patent Application Publication
(10) Pub. No.: US 2018/0028297 A1
(43) Pub. Date: Feb. 1, 2018

(54) UNBALANCED TONGUE-PRESSING OCCLUDER

(71) Applicant: Yen-Chang LIN, Nantou County (TW)

(72) Inventor: Yen-Chang LIN, Nantou County (TW)

(21) Appl. No.: 15/553,534

(22) PCT Filed: Feb. 26, 2015
(86) PCT No.: PCT/CN2015/073309

§ 371 Date: Aug. 24, 2017

(51) Int. Cl. A61C 19/06; A61F 5/56

(52) U.S. Cl. CPC A61C 19/06; A61F 5/56

(57) ABSTRACT

An unbalanced tongue-pressing occluder used in oral cavity includes a tongue depressing member, a first occluded member, and a second occluded member. The tongue depressing member resists against a tongue. The first occluded member and the second occluded member are connected with the tongue depressing member and occluded by a left teeth row and a right teeth row, respectively. The thickness of the first occluded member is different from the thickness of the second occluded member. Due to the thickness of the first occluded member differing from the thickness of the second occluded member, a height difference exists between the left teeth row and the right teeth row, such that the myofascia passing through the whole body produces a potential difference to cause a microcurrent variation, so as to alleviate the myofascial pain.

### BACKGROUND OF THE INVENTION

### 1. Field of the Invention

The present invention relates to brace structures, and more particularly, to an unbalanced tongue-pressing occluder.

### 2. Description of the Related Art

In a fast paced computerized age with huge working pressure, many people live a life suffering consistent muscle tightness, fixed posture, and working overtime. Therefore, myofascia is abnormally pressed, so as to greatly increase the possibility of myofascial pain occurrence. Although myofascial pain will not threat life of the patient, it does impose negative effect upon the sleeping quality, working efficiency, and life functions. Medication, rehabilitation, acupuncture, and intra-muscular electrical stimulation are considered as common method of treatment.

Myofascia is a connective tissue extending from head to toes, so as to cover the whole human body and form a stereoscopic network which surrounds every portions of the body from muscle, bone, nerve, blood vessel, organ, to cell levels. When the pressure or tension of any body tissue changes, the myofascia generates a bioelectricity that flows through the whole body with a speed which is three times higher than the transmitting speed of nerve. Acupuncture and massage apply such a fast transmitting stimulation to improve the myofascial pain symptoms of different body parts.

Acupuncture and intra-muscular electrical stimulation are common therapies for treating myofascial pain and tightness. However, such methods are invasive treating methods. The patient tends to panic during the treating process, even have a fainting spell during acupuncture treatment. As a result, the patient will be less willing to continue the treatment, failing to acquire a proper treating effect. Further, acupuncture and intra-muscular electrical stimulation shall be conducted by a skilled and professional practitioner or doctor, failing to achieve the convenience of treatment.

U.S. Pat. No. 8,761,874 “Electro-optical tissue stimulator and method of use” discloses an electro-optical stimulator, comprising a housing, a microcurrent electrostimulation therapy unit contained in the housing, and an optical radiation therapy unit. The user is able to hold the electro-optical stimulator by hand, thereby conducting a microcurrent electrostimulation by the microcurrent electrostimulation therapy unit, or conducting a light therapy by the optical radiation therapy unit. Therefore, the user is allowed to independently complete the treatment of myofascial pain without the need of other professional therapies.

The aforementioned electro-optical stimulator enables the user to complete the treating process of the myofascial pain. However, the electro-optical stimulator shall be applied upon the affected area, which requires a certain degree of privacy. Due to frequent public and social activities of modern people, the electro-optical stimulator is not suitable for providing a long term treatment. Therefore, the treating effect is compromised.

### SUMMARY OF THE INVENTION

The main objective of the present invention is to alleviate the myofascial pain with a non-invasive method.

For achieving the aforementioned objective, an embodiment of the present invention provides an unbalanced tongue-pressing occluder used in oral cavity, comprising a tongue depressing member, a first occluded member, and a second occluded member. The tongue depressing member resists against a tongue. The first occluded member and the second occluded member are connected with the tongue depressing member and occluded by a left teeth row and a right teeth row, respectively. The thickness of the first occluded member is different from the thickness of the second occluded member.

Based on the illustration above, the present invention includes following features.

With the tongue depressing member resisting against the tongue, soft tissue in the oral cavity is stably positioned, so as to prevent the tongue from being relaxed and falling backward during sleep, increase the muscle tension of the upper respiratory tract, and prevent the respiratory air flow from vibrating to stimulate the oral soft tissue. Therefore, symptoms such as snoring, sleep apnea syndrome, narrowing respiratory tract, tonsillitis, and adenoiditis are able to be improved.

Oxygen inhaled during breathing is a source of free radical in the human body, and oxygen free radial produced by breathing causes strong oxidization possibly induces aging and diseases. Therefore, with the tongue depressing member resisting against the tongue, symptoms caused by oral breathing and hyperpnea are further alleviated, including allergies such as allergic rhinitis and asthma, sleep disorders such as dreamy sleep, neural system dysfunction, digestion tract issues such as abdominal bloating and gastroesophageal reflux, autoimmune diseases, cardiovascular diseases, metabolism diseases, degeneration diseases, physical fatigue and aches, unknown pain of whole body, and cellular diseases of organs and tissues.

By use of the first occluded member and the second occluded member having different thicknesses, during the occlusion, a height difference exists between the left teeth row and the right teeth row. Therefore, the myofascia passing through the whole body generates slight potential difference accordingly, so as to produce a microcurrent variation. As a result, an alleviation is provided upon symptoms caused by myofascial pain, such as frozen shoulder, gao-huang pain (pain in the area of rhomboid muscle), tennis elbow, De Quervain's disease, trigger finger, tendonitis, plantar fasciitis, and sciatica.

### BRIEF DESCRIPTION OF THE DRAWINGS

FIG. 1 is a perspective view of the unbalanced tongue-pressing occluder in accordance with an embodiment of the present invention.

FIG. 2 is a top view of the unbalanced tongue-pressing occluder in accordance with an embodiment of the present invention.

FIG. 3A to FIG. 3B are schematic views illustrating the operation status of the embodiment of the present invention.

FIG. 4A to FIG. 4B are schematic views illustrating another operation status of the embodiment of the present invention.

### DETAILED DESCRIPTION OF THE INVENTION

The aforementioned technical features of the present invention will be understood by reference to the drawings as following.

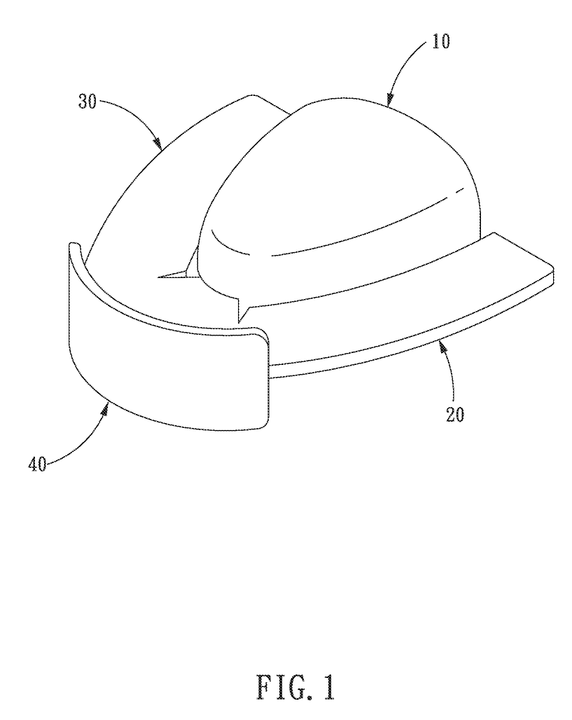

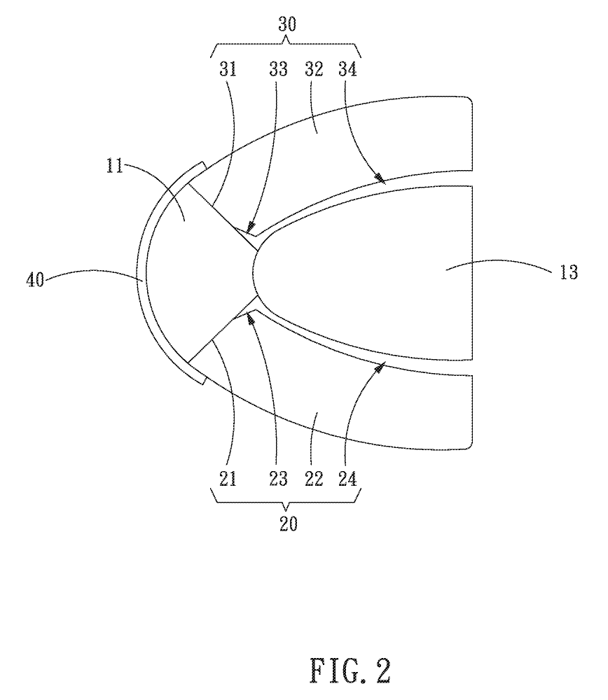

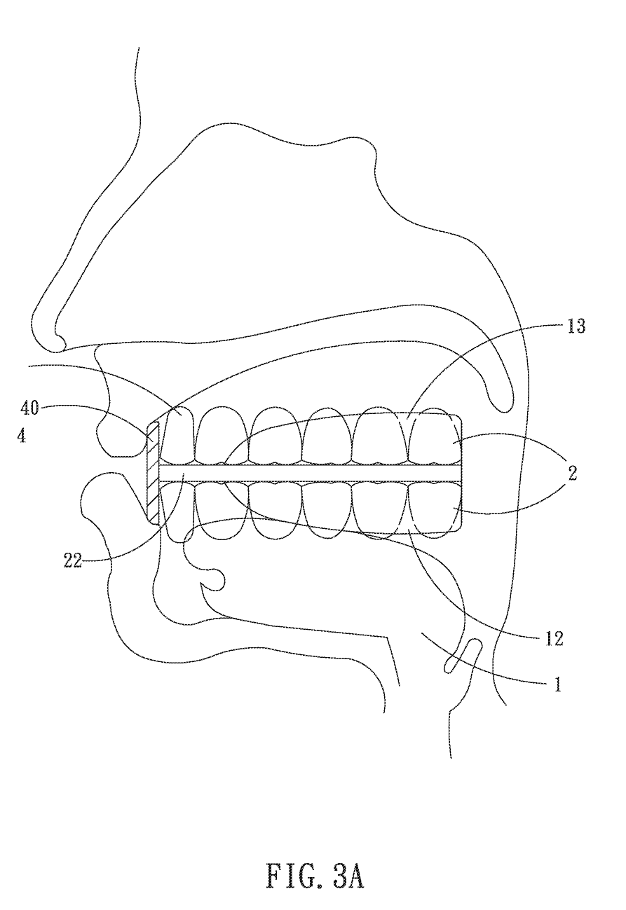

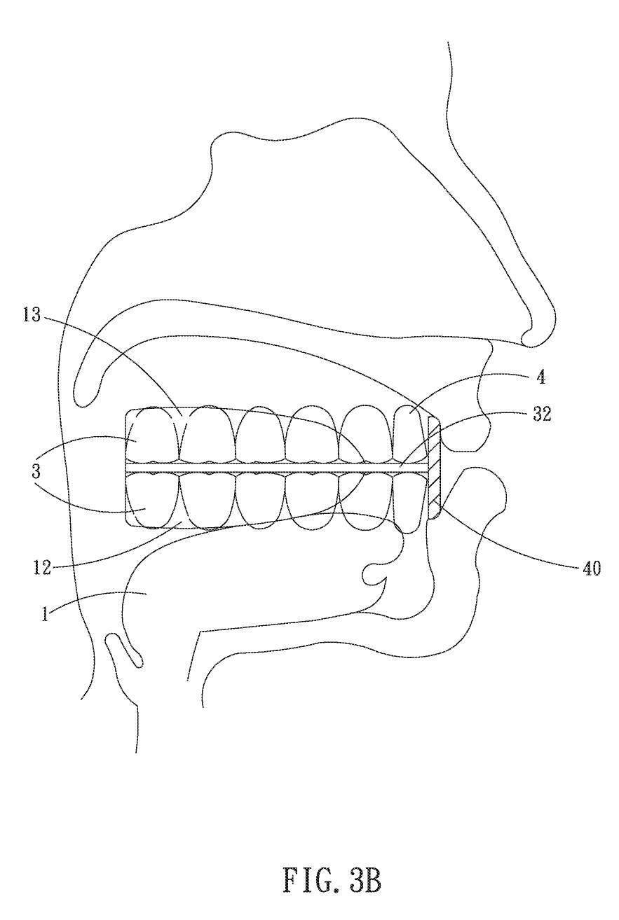

Referring to FIG. 1, FIG. 2, FIG. 3A, and FIG. 3B, an embodiment of unbalanced tongue-pressing occluder is provided, which is placed in the oral cavity and includes a tongue depressing member 10, a first occluded member 20, and a second occluded member 30. The tongue depressing member 10 resists against a tongue 1. The first occluded member 20 and the second occluded member 30 are connected with the tongue depressing member 10 and occluded by a left teeth row 2 and a right teeth row 3, respectively. Also, the thickness of the first occluded member 20 is different from the thickness of the second occluded member 30.

Further, the first occluded member 20 comprises a first connecting portion 21 and a first extension portion 22. The first connecting portion 21 is connected with the tongue depressing member 10. The first extension portion 22 is connected with the first connecting portion 21 and spaced with the tongue depressing member 10 by a first gap 24. The second occluded member 30 comprises a second connecting portion 31 and a second extension portion 32. The second connecting portion 31 is connected with the tongue depressing member 10. The second extension portion 32 is connected with the second connecting portion 31 and spaced with the tongue depressing member 10 by a second gap 34. With the first gap 24 and the second gap 34, the first occluded member 20 and the second occluded member 30 are provided with a buffering width, respectively. When the unbalanced tongue-pressing occluder is placed into the oral cavity, the inner sides of the cheeks inside the oral cavity push against the first extension portion 22 and the second extension portion 32 toward the first gap 24 and the second gap 34, respectively, so as to fit the different inner diameter of the oral cavity. Further, the first occluded member 20 includes a first buffer recess 23 disposed at the junction between the first connecting portion 21 and the tongue depressing member 10 and also connected with the first gap 24. The second occluded member 30 includes a second buffer recess 33 disposed at the junction between the second connecting portion 31 and the tongue depressing member 10 and also connected with the second gap 34. When the unbalanced tongue-pressing occluder is placed in the oral cavity, the first gap 24 and the second gap 34 are pushed and deformed by the oral cavity to be smaller, so as to possibly cause the discomfort of the user. However, by use of the first buffer recess 23 and the second buffer recess 33, the discomfort caused by the deformation of the first gap 24 and the second gap 34 is avoided.

In addition, the present invention further includes a front resisting member 40, and the tongue depressing member further includes a front occluded area 11, a first protrusion 12, and a second protrusion 13. The front occluded area 11 is configured to be occluded by a front teeth row 4, and the front resisting member 40 is disposed on one side of the front occluded area 11 away from the tongue 1. The front teeth row 4, when occluding the front occluded area 11, resists against the front resisting member 40.

Therefore, the unbalanced tongue-pressing occluder is stably positioned and prevented from accidentally sliding when being occluded by the user.

Noticeably, in the embodiment of the present invention, the first connecting portion 21 and the second connecting portion 31 are disposed in adjacent to the front resisting member 40 and connected with the front occluded area 11, such that the first gap 24 and the second gap 34 face the inner side of the oral cavity away from the front resisting member 40. However, the present invention is not limited to such disposition. The first connecting portion 21 and the second connecting portion 31 are allowed to be connected with the positions of the tongue depressing member 10 away from the front resisting member 40, such that the first gap 24 and the second gap 34 face the front resisting member 40. In such arrangement, the first extension portion 22 and the second extension portion 32 are also able to be adjusted according to different size of the oral cavity.

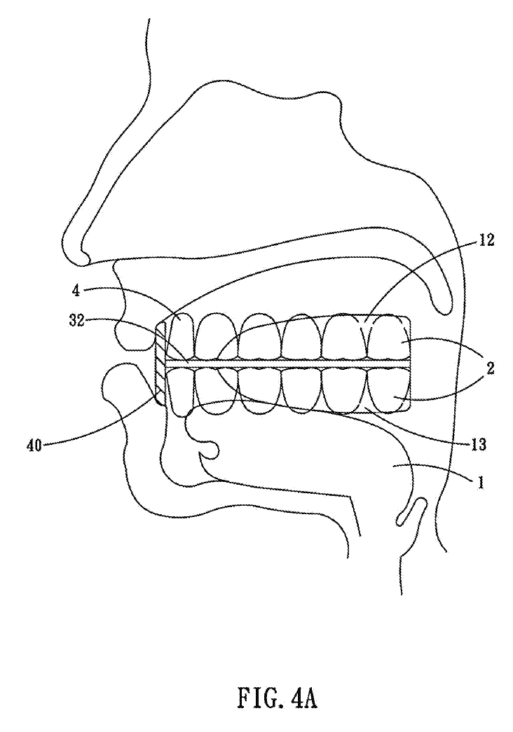

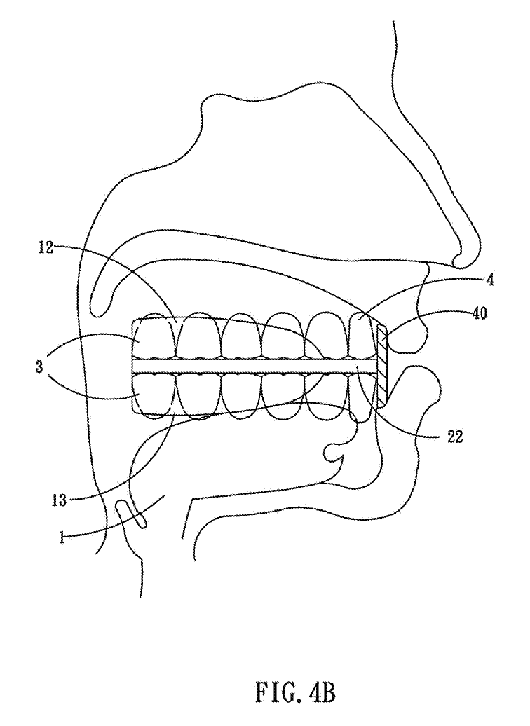

The first protrusion 12 extends to form toward the tongue 1, and the second protrusion 13 extends to form away from the first protrusion 12. Referring to FIG. 3A and FIG. 3B, in a preferred embodiment of the present invention, the user uses the left teeth row 2 to occlude the first occluded member 20, and uses the right teeth row 3 to occlude the second occluded member 30, such that the first protrusion 12 of the tongue depressing member 10 resists against the tongue 1. Meanwhile, due to the thickness difference between the first occluded member 20 and the second occluded member 30, the myofascia generates a potential difference to cause a current variation, thereby alleviating the myofascial pain. However, when the myofascia adapts the thickness difference between the first occluded member 20 and the second occluded member 30, the potential difference is no longer produced, so as to lose the alleviation of myofascial pain. At this time, the user needs to simply turn the unbalanced tongue-pressing occluder upside down. Referring to FIG. 4A and FIG. 4B, in another preferred embodiment of the present invention, the user uses the left teeth row 2 to occlude the second occluded member 30, and uses the right teeth row 3 to occlude the first occluded member 20, such that the second protrusion 13 of the tongue depressing member 10 resists against the tongue 1. Therefore, the thickness occluded by the left teeth row 2 and the right teeth row 3 varies again, whereby the myofascia produces another potential difference to cause a current variation. Therefore, the alleviation of myofascial pain is achieved again.

To sum up, the present invention achieves following technical features.

With the tongue depressing member resisting against the tongue, soft tissue in the oral cavity is stably positioned, so as to prevent the tongue from being relaxed and falling backward during sleep, increase the muscle tension of the upper respiratory tract, and prevent the respiratory air flow from vibrating to stimulate the oral soft tissue. Therefore, symptoms such as snoring, sleep apnea syndrome, narrowing respiratory tract, tonsillitis, and adenoiditis are able to be improved.

Oxygen inhaled during breathing is a source of free radical in the human body, and oxygen free radial produced by breathing causes strong oxidization possibly induces aging and diseases. Therefore, with the tongue depressing member resisting against the tongue, symptoms caused by oral breathing and hyperpnea are further alleviated, including allergies such as allergic rhinitis and asthma, sleep disorders such as dreamy sleep, neural system dysfunction, digestion tract issues such as abdominal bloating and gastroesophageal reflux, autoimmune diseases, cardiovascular diseases, metabolism diseases, degeneration diseases, physical fatigue and aches, unknown pain of whole body, and cellular diseases of organs and tissues.

With the first occluded member and the second occluded member having different thicknesses, during the occlusion, a height difference exists between the left teeth row and the right teeth row. Therefore, the myofascia passing through the whole body generates slight potential difference accordingly, so as to produce a microcurrent variation for alleviating the myofascial pain. Different from external stimulation such as acupuncture and massage, oral occlusion applies the chewing muscle, which is able to provide the greatest and frequent effect upon the myofascia by self-control. Therefore, through the unbalanced occlusion difference between the left teeth row and the right teeth row, the bioelectricity is produced to stimulate the myofascia, so as to immediately adjust the unbalanced and clogged myofascia network. As a result, various myofascial pain syndromes, including the pain, tightness, or bulge of face, head, shoulder, scapula, waist, back, or limbs, are alleviated. Trigger points pains and referred pains thereby derived are able to be improved, so as to further alleviate various symptoms caused by myofascial pain, such as frozen shoulder, gao-huang pain (pain in the area of rhomboid muscle), tennis elbow, De Quervain's disease, trigger finger, tendonitis, plantar fasciitis, and sciatica.

With the first gap between the first extension portion and the tongue depressing member and the second gap between the second extension portion and the tongue depressing member, the first occluded member and the second occluded member are internally moved to provide a buffering, so as to fit different oral diameters of different users.

With the first buffer recess and the second buffer recess, the first extension portion and the second extension portion are prevented from deforming toward the tongue depressing member when being internally moved.

By use of the front resisting member, the unbalanced tongue-pressing occluder is stably positioned when being placed in the oral cavity.

With the first protrusion and the second protrusion, the unbalanced tongue-pressing occluder is allowed to be turned upside down for application, so as to adjust the occlusion thickness of the left teeth row and the right teeth row, thus preventing the myofascia from stopping producing potential difference after adapting the thickness difference

What is claimed is:

1. An unbalanced tongue-pressing occluder applied in an oral cavity, characterized in, the unbalanced tongue-pressing occluder comprising:
a tongue depressing member resisting against a tongue; and
a first occluded member and a second occluded member for left teeth row and a right teeth row, respectively, the first occluded member and the second occluded member connected with the tongue depressing member, a thickness of the first occluded member differing from a thickness of the second occluded member.

2. The unbalanced tongue-pressing occluder of claim 1, characterized in further including a front occluded area which is occluded by a front teeth row.

3. The unbalanced tongue-pressing occluder of claim 2, characterized in further including a front resisting member disposed on one side of the front occluded area away from the tongue, the front teeth row occluding the front occluded area and resisting against the front resisting member.

4. The unbalanced tongue-pressing occluder of claim 1, characterized in the first occluded member including a first connecting portion connected with the tongue depressing member, the first occluded member also including a first extension portion spaced with the tongue depressing member by a first gap, the second occluded member including a second connecting portion connected with the tongue depressing member, the second occluded member also including a second extension portion spaced with the tongue depressing member by a second gap.

5. The unbalanced tongue-pressing occluder of claim 4, characterized in the first connecting portion and the second connecting portion being connected to one end of the tongue depressing member in adjacent to a front teeth row, such that an opening of the first gap and an opening of the second gap are away from the front teeth row.

6. The unbalanced tongue-pressing occluder of claim 4, characterized in the first connecting portion and the second connecting portion being connected to one end of the tongue depressing member away from a front teeth row, such that an opening of the first gap and an opening of the second gap are in adjacent to the front teeth row.

7. The unbalanced tongue-pressing occluder of claim 4, characterized in the first occluded member further including a first buffer recess disposed at a junction between the first connecting portion and the tongue depressing member and connected with the first gap, the second occluded member further including a second buffer recess disposed at a junction between the second connecting portion and the tongue depressing member and connected with the second gap.

8. The unbalanced tongue-pressing occluder of claim 1, characterized in the tongue depressing member further including a first protrusion extending toward the tongue and a second protrusion extending away from the first protrusion.
